(19) United States
(12) Patent Application Publication
(10) Pub. No.: US 2024/0065266 A1
(43) Pub. Date: Feb. 29, 2024

(54) A METHOD OF OBTAINING A PREPARATION BASED ON OLIGOGALACTURONIDES AND THE USE THEREOF IN AGRICULTURE

(71) Applicant: INTERMAG SP. Z O.O., Olkusz (PL)

(72) Inventors: Hubert KARDASZ, Olkusz (PL); Krzysztof AMBROZIAK, Kraków (PL); Klaudia GASKA, Beblo (PL); Roksana RAKOCZY-LELEK, Czernichów (PL)

(21) Appl. No.: 18/259,882

(22) PCT Filed: Oct. 14, 2021
(86) PCT No.: PCT/PL2021/050072

§ 371 Date: Jun. 29, 2023

(30) Foreign Application Priority Data
Jan. 15, 2021 (PL) P.436671

(51) Int. Cl. A01N 43/16; A01P 3/00; A01P 7/04; A01P 21/00; C12P 19/04; C12P 19/14

(52) U.S. Cl. CPC A01N 43/16; A01P 3/00; A01P 7/04; A01P 21/00; C12P 19/04; C12P 19/14

(57) ABSTRACT

The present invention relates to the method for obtaining a preparation based on oligogalacturonides (OGA) with a degree of polymerization DP from 2 to 10 by an enzymatic method and the use thereof in agriculture or horticulture or vegetable cultivation, to biostimulate plant growth, enhance plant resistance to pathogens and pests, reduce plant fungal diseases and plant pests, especially in the cultivation of vegetables, horticultural plants and cereals.

### FIELD OF THE INVENTION

The object of the invention is a method of obtaining a preparation based on oligogalacturonides (OGA) and the use thereof in agriculture.

Oligogalacturonides are low molecular weight pectin derivatives that are usually obtained by depolymerization of natural high molecular weight pectins.

The best known method for obtaining this type of compounds is a method of enzymatic pectin hydrolysis using pectinases. There is also, among others, a pectin alkaline depolymerization method, a high-temperature hydrolysis method and methods of polyuronide depolymerization using an oxidizing agent.

Depending on the degree of polymerization, i.e. from 6 to 25-30 monosaccharide residues, oligogalacturonides have different properties and uses. For example, oligogalacturonides with degrees of polymerisation DP from 6 to 9 have been found to be capable of forming strong complexes with heavy metal ions such as lead, mercury, cadmium, and others. Oligogalacturonides with a higher degree of polymerization stimulate flower formation and the elongation of the apical meristems of the roots and shoots.

### BACKGROUND OF THE INVENTION

European patent EP1373543 B1 discloses a method for preparing pectin hydrolysis products comprising treating pectin or pectin-containing plant material in an aqueous solution or suspension with a pectin hydrolysing enzyme A in the first step, and a pectin hydrolysing enzyme B in the second step of the method, and using the products in oral pharmaceutical formulations. Pectin hydrolysis products prepared by the method of the invention have a fraction of galacturonides that contain at least one 4,5-unsaturated galacturonic acid molecule and that are esterified with methanol with ≥20% yield. Optionally, in the method of the disclosure, liquid hydrolysis products obtained from the second step of the method are treated with enzyme C in the third step of the method, wherein enzyme C is pectinesterase.

Spanish patent ES2537936 B1 discloses a method for preparing modified citrus pectins characterized in that the raw material derived from the by-products of the citrus industry is hydrated and subjected to enzymatic treatment with cellulase and pectinesterase under mild conditions. According to the invention, the method comprises preparing modified citrus pectin and an alcohol extract. It involves adding water and 5-10% sodium citrate to the by-products of citrus juice production, i.e. peel or pulp, in order to increase the pH. Enzymatic treatment with the cellulase solution is then performed, maintaining the product at the temperature of 40-50° C. for 30 minutes while stirring. The enzymatic treatment method is similarly repeated using pectinesterase under the same reaction conditions. After this step is completed, enzymatic deactivation is performed by heat treatment at about 90° C. for 1-5 minutes, then the solution is cooled to 25-30° C. and extracted with methanol at room temperature. The method according to the invention yields pectin with a molecular weight of 10-20 KDa and a degree of polymerization 30-70 units.

American patent U.S. Pat. No. 9,113,650 B1 discloses a method for preparing hydrolysed PET-pectin, comprising:
a) subjecting a citrus pectin solution to enzymatic hydrolysis using a mixed pectinase solution under acidic conditions at pH=4 and at the temperature from 45° C. do 65° C. for 1 hour to 72 hours, in order to prepare a pectin hydrolysate, wherein the citrus pectin solution has pectin concentration from 1 to 3% by volume and the mixed pectinase solution contains pectin methylesterase, polygalacturonase and pectin lyase, and the volume ratio of the mixed pectinase solution to the citrus pectin solution is 1000:1, and then
b) submitting the pectin hydrolysate to the heat treatment at the temperature of 100° C. for 10 minutes, in order to complete enzymatic hydrolysis.

The resulting pectin is completely hydrolysed and has an average molecular weight less than or equal to 1 kDa.

Russian patent RU2478649 Cl discloses a method for preparing low molecular weight pectin, comprising pectin hydrolysis in an aqueous mineral acid solution by heating in order to separate the liquid phase from the insoluble pectin residue, isolating the target hydrolysis products by precipitation with an organic solvent miscible with water. The feed material for the hydrolysis is pectin with a low degree of esterification, while the hydrolysis is carried out continuously in a drainage apparatus, maintaining the temperature at 70-100° C. The rate of feeding mineral acid into the chamber is calculated according to a specific formula. The prepared liquid phase after the hydrolysis process is subjected to additional heat treatment in a flow heat exchanger. The liquid phase is neutralized to a pH of at least 4 before precipitation of the pectin.

European patent EP/PL2115066T3 discloses a “bioactive” composition, which contains one or more oligogalacturonates ((1->4)-α-D-galacturonate) or any other oligosaccharides (oligoguluronates) that may exhibit the “egg box” conformation and is stabilized by one or more polycationic saccharides, preferably chitosan oligosaccharide or chitosan polysaccharide. According to the disclosure, the composition synergistically increases the biological activity of each oligosaccharide and combines their individual enhancing activities in diverse fields. For example, it can be an elicitor composition that is used to protect plants (to increase plant natural defence against pathogens) and to stimulate plant growth and differentiation. The composition according to the invention may also be a fertilizing composition used to increase, for example, plant yields through increased height, thickness (of stems, leaves, roots), biomass, or number of flowers/fruits per plant. In environmental applications, the composition disclosed in the invention can be used as a chelating agent (chelating heavy metal) or for wastewater treatment, especially in water treatment techniques, to segregate organic compounds and heavy metals. It can also be used to precipitate some waste compounds or other contaminating substances, such as DDT and polychlorobenzenes, or to fix radicals. Furthermore, the composition according to the invention may also readily be used in agricultural and agrochemical systems, as a preservative coating and biostatic agent when applied to fruit, vegetables and crops, as a fertilizer, as an agent increasing the number of useful soil microorganisms and reducing the number of harmful ones (“biological control”) or to stimulate the synthesis of protective agents by the plant itself, in order to accelerate germination and plant growth. The composition is used as a plant growth promoter, seed coating agent or antifungal adjuvant.

The changing climate, rapid changes in atmospheric conditions, drought periods alternating with heavy rainfall and the associated increased stress by diseases and pests are major problems in today's agriculture. Additionally, environmental pollution and the problem of pesticide residues in food of plant origin are all prompting a search for new solutions in the field of plant protection and stimulation, both in terms of adapting the plant to the changing atmospheric conditions and increasing its natural resistance to attacks by pests and disease-causing pathogens. Increased public awareness of healthy eating is forcing producers to seek and implement safe and biodegradable plant protection products into agricultural practice.

Prior art did not disclose any biostimulant preparations of natural origin that would combine improved plant defensive capabilities against pathogen and pest attacks with a simultaneous direct action on pests.

In the course of research and development, the inventors obtained a preparation with such properties.

### SUMMARY OF THE INVENTION

### Detailed Description of the Invention

The object of the invention is a method for obtaining a preparation based on oligogalacturonides (OGA) with a degree of polymerization DP from 2 to 10 by an enzymatic method, wherein water, a 40% tetrasodium ethylenediaminetetraacetate solution and sodium hydroxide are introduced into a reactor equipped with a stirrer and a heating system to obtain pH 12.0. Then fruit pectin is introduced into the reactor and the contents of the reactor are stirred until pectin has completely dissolved, then a 10% citric acid solution is added to obtain pH 5.0. Then a 0.1% pectinolytic enzyme solution is introduced into the reactor, after which the contents of the reaction mixture is heated to reach the temperature of 80° C., wherein the enzyme is deactivated. Heating is continued for another 30 minutes; then the reaction mixture is cooled to below 40° C. and a solution of potassium sorbate (1-5 kg of powder dissolved in a small amount of water) is added and stirred for another 30 minutes. The finished product in liquid form is poured into containers or spray-dried or subjected to the lyophilization process in order to obtain the product in solid form.

As used hereafter, the term “OGA preparation” means a preparation obtained by the method according to the present invention.

Preferably, in the method of the present invention, pectin is selected from apple or citrus pectin, particularly preferably citrus pectin is used.

The method of the invention uses a pectinolytic enzyme selected from the group comprising polygalacturonase or pectinase.

According to the method of the present invention, raw materials mainly of plant origin are used for preparing the OGA preparation, which is why the OGA preparation is safe for the environment during its use.

The object of the invention is also the use of the OGA preparation obtained by the method according to the present invention in agriculture or horticulture or vegetable cultivation as a preparation having a plant biostimulating activity and/or a contact activity on pests.

As shown in the embodiments, the OGA preparation of the present invention exhibits a biostimulating activity and improves plant condition by increasing their tolerance to changing atmospheric conditions. Moreover, the OGA preparation according to the present invention exhibits an activity stimulating plant acquired immunity by activating signalling pathways and genes responsible for plant defensive responses, which allows to reduce the use of chemical plant protection products, especially in horticultural and agricultural crops where plant-attacking pathogens or pests may cause damage to the entire crop. Therefore, the OGA preparation according to the present invention is preferably used in agriculture or horticulture or vegetable cultivation.

The OGA preparation according to the present invention can be considered a natural vaccine that supports plant immune processes. Additionally, this preparation has the effect of increasing, among others, sugar and polyphenol content in the plant, therefore, according to the present invention, the use of the OGA preparation can improve health-promoting properties of cultivated plants, especially fruits and vegetables, particularly those considered “superfoods”.

As shown in the embodiments, following the application of the OGA preparation of the present invention, increased useful crop yield and the quality of the yield, so the use of this preparation is indicated in the production of organic, healthy and safe food. According to the present invention, the OGA preparation is preferably used to enhance plant resistance to pathogens and pests or to reduce plant fungal diseases, especially in vegetable and cereal crops.

As shown in the embodiments, the OGA preparation according to the present invention has an ambivalent effect on plants, because, on the one hand, it stimulates the systemic acquired immunity of the plant, improving its defensive capabilities against the attack of pathogens and pests, and at the same time, in the case of a pest attack, it exhibits a contact activity, reducing the number of pests feeding on the infested plant. It is a unique property of the OGA preparation according to the present invention which is not known among other natural preparations of this type available in the state of the art. In the conducted experiments it was shown that the effectiveness of the OGA preparation according to the present invention is comparable to chemical preparations in certain applications. Therefore, the solution of the present invention is in accordance with the protection program of the Integrated Farm Management and with the principles of Organic Farming and may be an alternative to chemical preparations used in crops or it allows to reduce the amount of chemical preparations used, which may contribute to reducing the negative effects for man and the environment of using chemical plant protection products, especially those employed commonly and on a large scale in agricultural production.

Therefore, according to the present invention, it can be used as a preparation increasing resistance to the attack of pathogens, i.e. powdery and downy mildew, Alternaria, leaf rust, Septoria leaf spot of wheat, Fusarium cortical stem rot, Sharp eyespot and also to pest control in crops of useful plants, i.e. spider mites, thrips, aphids, as well as whitefly, diamondback moth and carrot fly. Particularly preferred effects against powdery mildew have been demonstrated, including, among others, Golovinomyces orontii, Oidium lycopersici, Erysiphe betae, Erysiphe heraclei species which attack mostly vegetable crops.

The invention is presented in the embodiments that do not limit its scope.

### Example 1. Obtaining the OGA Preparation

900 dm³ of water is introduced into a reactor equipped with a stirrer and a heating system, then the stirrer is switched on and 2.5 dm³ solution of 40% tetrasodium ethylenediaminetetraacetate and 2-5 kg sodium hydroxide are introduced respectively. After the raw materials have dissolved and after the addition of sodium hydroxide the target pH 12.0 of the mixture should be obtained.

Then 10 kg of apple pectin (Pectin, Centro-chem) is fed and stirred until completely dissolved, followed by the introduction of 20-25 kg of a 10% citric acid solution to the reactor to obtain pH 5.0. Then 2 dm³ of a 0.1% pectinolytic enzyme—pectinase solution (Rohapect®, AB Enzymes) is added to the reaction mixture and the whole is heated to the temperature of 80° C. which is maintained for another 30 minutes. The heating is then switched off and the reaction mixture is allowed to cool to the temperature below 40° C., then 1 kg of potassium sorbate in the form of a solution is added (dissolved previously in a small amount of water needed to completely dissolve the powder) and stirred for another 30 minutes. At the completion of the process water is added to the reactor to obtain 1000 dm³ of the total volume of the mixture.

After mixing is completed, the finished product in liquid form can be poured into containers or further processed (spray drying, lyophilization) to obtain a solid form.

The degree of polymerization of the OGA preparation was tested according to the procedure described in Example 6, the tests showed pectin polymerization at the DP level of 9±1.

### Example 2. Obtaining the OGA Preparation

900 dm³ of water is introduced into a reactor equipped with a stirrer and a heating system, then the stirrer is switched on and 2.5 dm³ solution of 40% tetrasodium ethylenediaminetetraacetate and 2-5 kg sodium hydroxide are introduced respectively. After the raw materials have dissolved and after the addition of sodium hydroxide the target pH 12.0 of the mixture should be obtained.

Then 10 kg of citrus pectin (Pectin Classic CU 201, Herbstreith) is introduced and stirred until completely dissolved, followed by the introduction of 20-25 kg of a 10% citric acid solution to the reactor to obtain pH 5.0. Then 6 dm³ of a 0.1% pectinolytic enzyme solution—polygalacturonase (Pectinex SP-L, Novozymes) is added to the reaction mixture and the whole is heated to reach the temperature of 80° C. which is maintained for another 30 minutes. The heating is then switched off and the reaction mixture is allowed to cool to the temperature of below 40° C., then 2.5 kg of potassium sorbate in the form of a solution is added (powder is previously dissolved in a small amount of water needed to completely dissolve the powder) and stirred for another 30 minutes. At the completion of the process water is added to the reactor to obtain 1000 dm³ of the total volume of the mixture.

After mixing is completed, the finished product in liquid form can be poured into containers or further processed (spray drying, lyophilization) to obtain a solid form.

The degree of polymerization of the OGA preparation was tested according to the procedure described in Example 6, the tests showed pectin polymerization at the DP level of 6±1.

### Example 3 Obtaining the OGA Preparation

900 dm³ of water is introduced into a reactor equipped with a stirrer and a heating system, then the stirrer is switched on and 2.5 dm³ solution of 40% tetrasodium ethylenediaminetetraacetate and 2-5 kg sodium hydroxide are introduced respectively. After the raw materials have dissolved and after the addition of sodium hydroxide the target pH 12.0 of the mixture should be obtained.

Then 10 kg of citrus pectin (Pectin Classic CU 201, Herbstreith) is introduced and stirred until completely dissolved, followed by the introduction of 20-25 kg of a 10% citric acid solution to the reactor to obtain pH 5.0. Then 10 dm³ of a 0.1% pectinolytic enzyme solution—polygalacturonase (Pectinex SP-L, Novozymes) is added to the reaction mixture and the whole is heated to reach the temperature of 80° C. which is maintained for another 30 minutes. The heating is then switched off and the reaction mixture is allowed to cool to the solution temperature of below 40° C., then 5 kg of potassium sorbate in the form of a solution is added (the powder is previously dissolved in a small amount of water needed to completely dissolve the powder) and stirred for another 30 minutes. At the completion of the process water is added to the reactor to obtain 1000 dm³ of the total volume of the mixture.

After mixing is completed, the finished product in liquid form can be poured into containers or further processed (spray drying, lyophilization) to obtain a solid form.

The degree of polymerization of the OGA preparation was tested according to the procedure described in Example 6, the tests showed pectin polymerization at the DP level of 3±1.

### Example 4 Obtaining the OGA Preparation in Solid Form

The solution obtained according to Examples 1-3 can be spray dried to yield the product in solid form containing OGA in the amount up to 10% (m/m).

Drying techniques known to a person skilled in the art are used under given conditions, where:
the inlet air supply temperature is 180-220° C.,
the outlet air supply temperature is 80-100° C.,
the inner bed reaches the inlet temperature of 75-90° C.,
the outer bed reaches the inlet temperature of 20-30° C.,
the solution flow rate through the system obtained according to Examples 1-3 is 1000-2000 kg/h.

The obtained DP values for the solid preparation have the values corresponding to their liquid stock solutions.

### Example 5

The solution obtained according to Examples 1-3 can undergo lyophilization to yield the product in solid form containing OGA in the amount up to 10% (m/m).

A lyophilizer is used in order to obtain the material in the form of a lyophilizate.

Lyophilization techniques known to a person skilled in the art are used under given conditions, where:
the solution obtained according to Examples 1-3 is frozen to the temperature from −35 to −25° C., maintaining such conditions for 2 hours,
the drying process is carried out under the pressure of 0.2 mbar at 20-30° C. for 20 hours,
the obtained material is dried at 30-40° C. under the pressure of 0.1 mbar for 1-2 hours.
the obtained DP values for the preparation in the form of a lyophilizate have the values corresponding to their liquid stock solutions.

### Example 6

### Study of the Degree of Polymerisation (DP) Range in the OGA Preparation

Study of the degree of polymerisation of the OGA preparation obtained by the method according to the present invention were performed for the fluid and solid form of the preparation. The method for determining the galacturonic acid content using the spectrophotometric technique with m-hydroxydiphenyl according to Blumenkrantz, Asboe-Hansen method. Under assay conditions, complete hydrolysis of pectin substances to galacturonic acids (Gala) occurs. These acids react with sulfuric acid to form 5-formyl-2-furancarboxylic acid (5FF). In turn, these derivatives react with m-hydroxydiphenyl (MHDP) to form pink-coloured complexes, with a maximum absorbance at 520 nm wavelength. The Bertrand method was used to determine the content of reducing ends of galacturonic acid. The oligosaccharide assay is performed by an indirect method on the basis of the amount of potassium permanganate (VII) solution used to titrate Fe⁺² ions corresponding stoichiometrically to the amount of reducing polysaccharides contained in the test solution. Three Bertrand fluids (I— copper (II) sulphate (VI); II— potassium and sodium tartrate and sodium hydroxide; III— iron (III) sulphate (VI) in concentrated sulphuric acid (VI)) are used in this method. The assay involves a quantitative reduction of Cu⁺² ions to Cu⁺¹ by polysaccharides containing free reducing groups in the molecule which occurs in a strongly alkaline environment and at the boiling point of the solution. In order to obtain a DP value, the ratio of the total galacturonic acid content determined by the Blumenkrantz, Asboe-Hansen (1973) method to the total content of the reducing ends determined by the Bertrand method should be compared. The above-mentioned relationship is determined using the formula:

$$Degree of polymerisation ( DP ) \frac{\begin{matrix}total amount of \\ galacturonic acid ( \frac{mg}{g} )\end{matrix}}{\begin{matrix}total amount of \\ free reducing ends ( \frac{mg}{g} )\end{matrix}}$$

### Example 7

### Study of the OGA Preparation Properties

Studies of the properties of the OGA preparation obtained by the method according to the present invention were performed for the liquid form of the preparation.

Experiments were conducted on plants grown in the greenhouse and in the field exposed to infestation by commonly occurring fungal diseases. The effect of the OGA preparation on the most common plant fungal diseases caused by powdery mildew and Alternaria was observed by dosing the OGA preparation of the present invention in a 2 L and 3 L dose per hectare of crops. The amount of the working solution in the experiments was 600 L/ha. For comparative purposes, control was used, (meaning plants without the application of the preparations) and a combination using commercially available plant protection products.

The OGA preparation according to the present invention was applied foliarly several times in 7-10 day intervals by spraying the plants. Doses of the preparations are given in the Tables with the results of individual experiments. OGA was used before the occurring of the symptoms of the disease in order to activate plant defensive processes. The effectiveness of the preparations was assessed on the basis of the degree of leaf infestation by powdery mildew or Alternaria in 7-10 day intervals. Experiments were conducted on cucumber and tomato crops in the greenhouse and field cultivation of beetroot, carrots and cabbage.

In the greenhouse cucumber crops, the effectiveness of OGA in both tested doses, observed 10 days after the treatment 2, was over 80% with 1.54% infestation on the control object. Further observations showed that the effectiveness was maintained at more than 80% for the 3 L/ha dose, while for the 2 L/ha dose it decreased to 76%. An increase in infestation to 19.3% in control plants (10 days after the treatment 4), and then to 39.6% (10 days after the treatment 5) caused the effectiveness of OGA to drop below 70% regardless of the dose (observation carried out 10 days after treatment 4), and then below 60%. Observations of the effectiveness of the OGA preparation in reducing powdery mildew Golovinomyces orontii in sheltered cucumber crops are presented in Table 1. The first OGA application occurred in the BBCH 16 phase.

TABLE 1
Effectiveness of the OGA preparation in reducing cucumber powdery
mildew (Golovinomyces orontii) in sheltered crops.

| | Observations | | | | | | | |
|---|---|---|---|---|---|---|---|---|
| | 10 days after | | 10 days after | | 10 days after | | 10 days after | |
| | treatment 2 | | treatment 3 | | treatment 4 | | treatment 5 | |
| COMBINATION | % leaf infestation/% effectiveness | | | | | | | |
| CONTROL | 1.54 | — | 5.3 | — | 19.3 | — | 39.6 | — |
| OGA 2 L/ha | 0.24 | 84 | 1.3 | 76 | 7.7 | 60 | 19.3 | 51 |
| (Example 2) | | | | | | | | |
| OGA 3 L/ha | 0.17 | 89 | 1.0 | 81 | 6.2 | 68 | 16.1 | 59 |
| (Example 2) | | | | | | | | |
| OGA 2 L/ha | 0.18 | 70 | 1.1 | 69 | 8.9 | 51 | 19.0 | 52 |
| (Example 1) | | | | | | | | |
| Scorpion 325 SC | 0.0 | 100 | 0.0 | 100 | 0.0 | 100 | 0.04 | 99 |
| (active substance: | | | | | | | | |
| Azoxystrobin, | | | | | | | | |
| Difenoconazole, | | | | | | | | |
| manufacturer: | | | | | | | | |
| Syngenta) | | | | | | | | |

In greenhouse tomato crops, the OGA preparation according to the present invention applied in tomato crops (first application in the BBCH 53-61 phase) effectively reduced the development of powdery mildew, its effectiveness observed 10 days after application 3 was 90% regardless of the dose applied. An increase in the infestation to 30.2% on the control object resulted in a decrease in the effectiveness, however, treatment 4 with the preparation allowed to maintain the effectiveness of the preparation at the level from 65% for the 3 L/ha dose to 75% for the 2 L/ha dose, the results are shown in Table 2.

TABLE 2
Effectiveness of the OGA preparation in reducing tomato powdery
mildew (Oidium lycopersici) in sheltered crops.

| | Observations | | | | | |
|---|---|---|---|---|---|---|
| | 10 days after | | 14 days after | | 7 days after | |
| | treatment 3 | | treatment 4 | | treatment 5 | |
| COMBINATION | % leaf infestation/% effectiveness | | | | | |
| CONTROL | 8.78 | — | 30.2 | — | 68.8 | — |
| OGA 2 L/ha | 0.78 | 91 | 7.4 | 75 | 36.6 | 46 |
| (Example 2) | | | | | | |
| OGA 3 L/ha | 0.84 | 90 | 10.4 | 65 | 42.9 | 37 |
| (Example 2) | | | | | | |
| OGA 2 L/ha | 2.08 | 76 | 18.1 | 40 | 48.9 | 29 |
| (Example 1) | | | | | | |
| Scorpion 325 S.C. | 0.0 | 100 | 0.0 | 100 | 0.2 | 99 |
| (active substance: | | | | | | |
| Azoxystrobin, | | | | | | |
| Difenoconazole, | | | | | | |
| manufacturer: | | | | | | |
| Syngenta) | | | | | | |

### Conclusions:

The foliar application of the OGA preparation in sheltered cucumber and tomato crops effectively reduced plant infestation by powdery mildew. OGA used before the occurring of the symptoms of the disease stimulates defensive processes and increases plant resistance. A repeat application of the OGA preparation maintains high effectiveness in reducing the powdery mildew development in sheltered vegetable crops.

In field beetroot crops (Table 3), observations of leaf infestation by the pathogen carried out 10 days after application 1 of OGA (BBCH 42-45 phase) showed its effectiveness at both doses tested at the level of approximately 70%. A repeat treatment allowed to reduce the infestation by powdery mildew and obtain effectiveness at the level of more than 50% on day 7 after application 2 and more than 40% on day 7 after application 3 (Table 3). The effectiveness in reducing powdery mildew in carrot crops after application 4 of the OGA preparation was high and was about 80% for the 2 L/ha and 3 L/ha dose. Observations carried out 7 and 14 days after treatment 5 showed effectiveness at the level of about 40% for the 2 and 3 L/ha dose (Table 4). The first OGA application in carrot crops occurred in the BBCH 42 phase.

TABLE 3
Effectiveness of the OGA preparation in reducing beetroot powdery
mildew (Erysiphe betae) in field crops.

| | Observations | | | | | |
|---|---|---|---|---|---|---|
| | 10 days after | | 7 days after | | 7 days after | |
| | treatment 1 | | treatment 2 | | treatment 3 | |
| COMBINATION | % leaf infestation/% effectiveness | | | | | |
| CONTROL | 9.7 | — | 45.5 | — | 72.5 | — |
| OGA 2 L/ha | 2.7 | 72 | 18.5 | 59 | 38.8 | 46 |
| (Example 3) | | | | | | |
| OGA 3 L/ha | 3.0 | 69 | 21.9 | 51 | 42.1 | 41 |
| (Example 3) | | | | | | |
| OGA 2 L/ha | 5.8 | 40 | 31.3 | 43 | 52.9 | 30 |
| (Example 1) | | | | | | |
| Scorpion 325 SC | 0.0 | 100 | 1.1 | 97 | 2.2 | 97 |
| (active substance: | | | | | | |
| Azoxystrobin, | | | | | | |
| Difenoconazole, | | | | | | |
| manufacturer: | | | | | | |
| Syngenta) | | | | | | |

TABLE 4
Effectiveness of the OGA preparation in reducing carrot powdery
mildew (Erysiphe heraclei) in field crops.

| | Observations | | | | | |
|---|---|---|---|---|---|---|
| | 7 days after | | 7 days after | | 14 days after | |
| | treatment 4 | | treatment 5 | | treatment 5 | |
| COMBINATION | % leaf infestation/% effectiveness | | | | | |
| CONTROL | 0.66 | — | 5.9 | — | 33.2 | — |
| OGA 2 L/ha | 0.13 | 80 | 2.6 | 56 | 20.1 | 40 |
| (Example 3) | | | | | | |
| OGA 3 L/ha | 0.14 | 78 | 3.2 | 46 | 22.6 | 32 |
| (Example 3) | | | | | | |
| OGA 2 L/ha | 0.25 | 57 | 4.7 | 27 | 26.7 | 20 |
| (Example 1) | | | | | | |
| Luna Experience | 0.0 | 100 | 0.0 | 100 | 0.03 | 99 |
| (active substance: | | | | | | |
| fluopyram, | | | | | | |
| tebuconazole, | | | | | | |
| manufacturer: | | | | | | |
| Bayer) | | | | | | |

OGA was also tested in the cucumber crops in order to determine the effect on downy mildew (Table 5). The effectiveness of OGA in reducing fungal-like organisms (downy mildew) in field crops was about 60% with the infestation of 5% on the control object. An increase in leaf infestation to 49.1%, resulted in a decrease in the effectiveness of OGA to below 40%. Subsequent observations showed a further large decrease in the effectiveness of OGA. The first application of the preparations occurred in the BBCH 65 phase.

TABLE 5
Effectiveness of the tested preparations in reducing the development of
cucumber downy mildew (Pseudoperonospora cubensis).

| | Observations: % leaf infestation/% | | | | | |
|---|---|---|---|---|---|---|
| | effectiveness | | | | | |
| | 7 days after | | 7 days after | | 7 days after | |
| COMBINATION | treatment 1 | | treatment 2 | | treatment 3 | |
| CONTROL | 5.0 | — | 49.1 | — | 79.5 | — |
| OGA 2 L/ha (Example 2) | 2.0 | 60 | 36.0 | 27 | 73.3 | 8 |
| OGA 3 L/ha (Example 2) | 1.7 | 66 | 32.3 | 34 | 65.7 | 17 |
| OGA 2 L/ha (Example 1) | 3.2 | 36 | 44.7 | 9 | 70.0 | 12 |
| Cabrio duo | 0.0 | 100 | 6.4 | 87 | 29.6 | 63 |
| (active substance | | | | | | |
| pyraclostrobin, dimethomorph, | | | | | | |
| manufacturer BASF) | | | | | | |

### Conclusions:

The foliar application of the OGA preparation in carrot and beetroot crops under field conditions effectively reduced plant infestation by powdery mildew. OGA used before the occurring of the pathogen infection stimulates plant defensive processes. OGA is effective in field crops, particularly in reducing the infestation by powdery mildew.

In field carrot and cabbage crops, the effectiveness of the OGA preparation in reducing the development of carrot leaf blight (Table 6) and cabbage black spot of crucifers (Table 7) was similar. In carrot crops, the first assessment performed showed the effectiveness of OGA at the level from 36% for the dose of 2 L/ha to 52% for 3 L/ha. At the 2nd assessment, OGA showed the effectiveness from 23 to 45%, and at the 3rd assessment from 21 to 41% (Table 6). First application in carrot crops was in the BBCH 42 phase.

On the day of the first assessment, the effectiveness in reducing cabbage leaves infestation by Alternaria spp. was 45% for OGA 3 L/ha, at subsequent assessments the effectiveness was about 30%. The effectiveness of OGA 2 L/ha was several percent lower (Table 7). The first OGA application in carrot crops was in the BBCH 41-42 phase.

TABLE 6
Effectiveness of the preparations in reducing the development of
carrot leaf blight (Alternaria dauci)

| | Observations | | | | | |
|---|---|---|---|---|---|---|
| | % leaf infestation/% effectiveness | | | | | |
| | 10 days after | | 7 days after | | 7 days after | |
| COMBINATION | treatment 2 | | treatment 3 | | treatment 4 | |
| CONTROL | 8.4 | — | 17.6 | — | 30.9 | — |
| OGA 2 L/ha | 5.4 | 36 | 13.5 | 23 | 24.3 | 21 |
| (Example 2) | | | | | | |
| OGA 3 L/ha | 4.0 | 52 | 9.7 | 45 | 18.2 | 41 |
| (Example 2) | | | | | | |
| OGA 2 L/ha | 5.1 | 39 | 13.9 | 21 | 25.3 | 18 |
| (Example 1) | | | | | | |
| Luna Experience | 0.4 | 95 | 1.0 | 94 | 2.2 | 93 |
| 400 S.C. active | | | | | | |
| substance: | | | | | | |
| fluopyram 200 g/L, | | | | | | |
| tebuconazole 200 | | | | | | |
| g/L, manufacturer: | | | | | | |
| Bayer | | | | | | |

TABLE 7
Effectiveness of the preparations in reducing the development of
cabbage black spot of crucifers (Alternaria spp.)

| | Observations | | | | | |
|---|---|---|---|---|---|---|
| | % leaf infestation/% effectiveness | | | | | |
| | 10 days after | | 7 days after | | 10 days after | |
| COMBINATION | treatment 2 | | treatment 3 | | treatment 4 | |
| CONTROL | 1.93 | — | 4.87 | — | 7.27 | — |
| OGA 2 L/ha (Example 2) | 1.13 | 42 | 3.39 | 30 | 5.42 | 25 |
| OGA 3 L/ha (Example 2) | 1.07 | 45 | 3.18 | 35 | 5.14 | 29 |
| OGA 2 L/ha (Example 1) | 1.2 | 38 | 3.48 | 29 | 5.5 | 24 |
| Signum 33 WG active | 0.0 | 100 | 0.16 | 97 | 0.25 | 97 |
| substance: boscalid, | | | | | | |
| pyraclostrobin | | | | | | |
| manufacturer: BASF SE | | | | | | |

### Conclusions:

The OGA preparation reduces the development of fungal diseases caused by fungi of the genus Alternaria in vegetable crops at the level of 40-50%. OGA is effective in protecting plants grown in the field.

An experiment was conducted in order to check the effectiveness of the OGA preparation in reducing fungal diseases in winter wheat.

In wheat crops, the OGA preparation was applied twice foliarly in the BBCH 30-31 and 49-55 phases at the dose of 2 L/ha (Table 8). Observations carried out after the second treatment on the subflag and flag leaf showed the effectiveness of the OGA preparation of over 40% in reducing powdery mildew and Septoria in winter wheat. The effectiveness in reducing the Fusarium cortical steam rot in wheat was at the level of 51%. On the other hand, the leaf rust on the subflag and flag leaves and sharp eyespot on the stems as a result of the OGA application were reduced in 60% (Table 8). In the case of leaf rust and Septoria leaf spot, the effectiveness of OGA was several percent lower than that of the chemical preparation.

TABLE 8
Effectiveness of the OGA preparation in reducing fungal diseases in winter wheat.

| | OBSERVATIONS | | | | |
|---|---|---|---|---|---|
| | | 29 days after the | | 24 days after the | |
| | 19 days after the | treatment 2, the subflag | | treatment 2, stalk | |
| | treatment 2, the subflag | leaf and the flag leaf | | | Sharp |
| | leaf and the flag leaf | Leaf rust | Septoria leaf spot | Fusarium cortical | eyespot |
| | Powdery mildew | (Puccinia | (Zymoseptoria | steam rot | (Rhizoctonia |
| | (Bloomeria graminis) | recondita) | tritici) | (Fusarium spp.) | crealis) |
| COMBINATION | Effectiveness (%) | | | | |
| CONTROL | — | — | — | — | — |
| OGA 2 L/ha | 40-43 | 45-60 | 31-42 | 51 | 60 |
| (Example 2) | | | | | |
| OGA 2 L/ha | 16-21 | 19-24 | 15-20 | 19 | 33 |
| (Example 1) | | | | | |
| Talius 200EC | 81-92* | 64-88* | 55-73* | 74* | 72* |
| (active substance | | | | | |
| proquinazid, | | | | | |
| manufacturer: | | | | | |
| DuPont) | | | | | |
| Prosaro 250EC | | | | | |
| (active substance: | | | | | |
| prothioconazole, | | | | | |
| tebuconazole, | | | | | |
| manufacturer: | | | | | |
| Bayer) | | | | | |

*Effectiveness read after the application of Talius 200EC (BBCH 30-31 phase) and Prosaro 250EC (49-55 phase)

### Conclusions:

The OGA preparation reduces the development of fungal diseases of cereal plants at the level of 40-60%.

A series of experiments were conducted to assess the impact of OGA on plant protection against commonly occurring pests. The OGA preparation was used according to the present invention at the dose of 2 L and 3 L per hectare of crops, 600 L/ha of the working solution was used. For comparative purposes, control was used, meaning crops without the application of the preparations and a combination using commercially available plant protection products.

The OGA preparation according to the present invention was applied twice foliarly in 7 day intervals by spraying the plants. In the presented studies, the first application took place after exceeding the harmful threshold for a given pest, in accordance with the EPPO (European and Mediterranean Plant Protection Organization https://www.eppo.int) standards. Doses of the preparations are given in the Tables together with the results.

Experiments were conducted on strawberry, cucumber, tomato, pepper, cabbage and carrot crops. The OGA preparation was applied foliarly in 2 L/ha and 3 L/ha doses and the effect of the OGA preparation on stinging and sucking pests, i.e. spider mites, thrips and aphids was studied. These pests suck the juices out of plant cells, thereby damaging plant tissues and causing losses in plant crops. The effectiveness (%) of pest control was calculated using the Henderson-Tilton formula which takes into account the size of the pest population before and after the treatment.

Henderson-Tilton Formula:Effectiveness (%)=(1−(K1×T2)/(K2×T1))×100,

where:
K1— number of pests before the treatment on the control plot
K2— number of pests after the treatment on the control plot
T1— number of pests before the treatment on the treated plot
T2— number of pests on the treated plot

The effectiveness of OGA in combating mobile forms of spider mites (Table 9) in the 2 L/ha and 3 L/ha dose, observed on day 7 after application 1 (T1+7), was more than 70% in strawberry and cucumber crops. Observation carried out on day 7 after the second application (T2+7) showed a decrease of a few percent in the effectiveness, however, after another 7 days, the effectiveness of OGA 2 L/ha increased to 80% for the strawberry and 70% for the cucumber (Table 9). The highest effectiveness in spider mite egg control (Table 10) of 91% was observed 14 days after treatment 2 (T2+14) with the OGA preparation in the 2 L/ha dose in strawberry crops and 69% for the 2 L/ha dose for cucumber. The effectiveness of the OGA preparation in the 2 L/ha dose in spider mite control was comparable to the synthetic standard product Vertigo (Table 10).

TABLE 9
Effectiveness in controlling two-spotted spider mite (Tetranychus
urticae) adult forms in strawberry and cucumber crops

| | EFFICACY (%) OF SPIDER MITE CONTROL (MOBILE FORMS) | | | | | | | | | |
|---|---|---|---|---|---|---|---|---|---|---|
| | Strawberry | | | | | Cucumber | | | | |
| COMBINATION | T1 + 3 | T1 + 7 | T2 + 7 | T2 + 14 | T2 + 21 | T1 + 3 | T1 + 7 | T2 + 7 | T2 + 14 | T2 + 21 |
| CONTROL | — | — | — | — | — | — | — | — | — | — |
| OGA 2 | 64 | 75 | 69 | 80 | 59 | 76 | 77 | 63 | 70 | 46 |
| L/ha | | | | | | | | | | |
| (Example | | | | | | | | | | |
| 2) | | | | | | | | | | |
| OGA 3 | 63 | 70 | 66 | 74 | 63 | 65 | 72 | 42 | 37 | 68 |
| L/ha | | | | | | | | | | |
| (Example | | | | | | | | | | |
| 2) | | | | | | | | | | |
| OGA 2 | 44 | 40 | 46 | 31 | 35 | 44 | 38 | 35 | 30 | 29 |
| L/ha | | | | | | | | | | |
| (Example | | | | | | | | | | |
| 1) | | | | | | | | | | |
| VERTIGO | 66 | 71 | 95 | 47 | 83 | 76 | 83 | 90 | 45 | 63 |
| (active | | | | | | | | | | |
| substance: | | | | | | | | | | |
| abamectin, | | | | | | | | | | |
| manufacturer: | | | | | | | | | | |
| Makhteshim | | | | | | | | | | |
| Agan) | | | | | | | | | | |

TABLE 10
Effectiveness in controlling two-spotted spider mite (Tetranychus
urticae) eggs in strawberry and cucumber crops

| | EFFICACY (%) OF SPIDER MITE CONTROL (EGGS) | | | | | | | | | |
|---|---|---|---|---|---|---|---|---|---|---|
| | Strawberry | | | | | Cucumber | | | | |
| COMBINATION | T1 + 3 | T1 + 7 | T2 + 7 | T2 + 14 | T2 + 21 | T1 + 3 | T1 + 7 | T2 + 7 | T2 + 14 | T2 + 21 |
| CONTROL | — | — | — | — | — | — | — | — | — | — |
| OGA 2 L/ha | 55 | 72 | 50 | 91 | 45 | 57 | 52 | 63 | 69 | 54 |
| (Example 3) | | | | | | | | | | |
| OGA 3 L/ha | 31 | 29 | 31 | 77 | 0 | 57 | 76 | 34 | 32 | 78 |
| (Example 3) | | | | | | | | | | |
| OGA 2 L/ha | 29 | 25 | 20 | 18 | 15 | 46 | 40 | 38 | 35 | 20 |
| (Example 1) | | | | | | | | | | |
| VERTIGO (active | 68 | 91 | 88 | 81 | 71 | 71 | 79 | 91 | 64 | 74 |
| substance: | | | | | | | | | | |
| abamectin, | | | | | | | | | | |
| manufacturer: | | | | | | | | | | |
| Makhteshim | | | | | | | | | | |
| Agan) | | | | | | | | | | |

The effectiveness of the OGA preparation against thrips in cucumber crops was the highest in relation to the nymph stage and it was 90% on day 21 after application 2 (T2+21) for the dose of 2 L/ha. Observations for this developmental stage carried out on day 7 and 14 after application 2 also showed high effectiveness of more than 80%. The highest effectiveness in relation to adult individuals was found 14 days after application 2 and it was 72% (Table 11). In tomato crops, the OGA preparation was also more effective in controlling nymphs as compared to adult individuals, which was more than 90%, 7 days after treatment 1 and 7 and 14 days after treatment 2. The 2 L/ha dose of the OGA preparation is more effective in controlling both adult individuals and nymphs. Observations carried out 14 days after treatment 2 showed that the OGA preparation in the 2 L/ha dose is 72% effective. In pepper crops, the effectiveness in controlling adult individuals was the highest for the OGA preparation in the 3 L/ha dose and was 100% seven days after treatment 2, the effectiveness in controlling thrips nymphs was maintained at the level of more than 50% 7 days after application 1 and 2 (Table 11).

TABLE 11
Effectiveness in controlling Western thrips (Frankliniella
occidentalis) in cucumber and tomato crops and Tobacco thrips (Thrips tabaci) in
pepper crops

| | ADULT SPECIMENS | | | | | NYMPHS | | | | |
|---|---|---|---|---|---|---|---|---|---|---|
| COMBINATION | T1 + 3 | T1 + 7 | T2 + 7 | T2 + 14 | T2 + 21 | T1 + 3 | T1 + 7 | T2 + 7 | T2 + 14 | T2 + 21 |
| | Effectiveness (% ) of the preparations in cucumber crops | | | | | | | | | |
| CONTROL | — | — | — | — | — | — | — | — | — | — |
| OGA 2 L/ha | 69 | 43 | 68 | 72 | 62 | 63 | 18 | 83 | 82 | 90 |
| (Example 2) | | | | | | | | | | |
| OGA 3 L/ha | 70 | 42 | 53 | 51 | 30 | 76 | 13 | 82 | 72 | 85 |
| (Example 2) | | | | | | | | | | |
| OGA 2 L/ha | 30 | 35 | 29 | 25 | 20 | 55 | 28 | 49 | 44 | 40 |
| (Example 1) | | | | | | | | | | |
| MOSPILAN 20 | 63 | 24 | 28 | 0 | 20 | 74 | 47 | 88 | 66 | 91 |
| EC | | | | | | | | | | |
| (active substance: | | | | | | | | | | |
| Acetamiprid, | | | | | | | | | | |
| manufacturer: | | | | | | | | | | |
| Sumi Agro) | | | | | | | | | | |
| | Effectiveness (% ) of the preparations in tomato crops | | | | | | | | | |
| CONTROL | — | — | — | — | — | — | — | — | — | — |
| OGA 2 L/ha | 45 | 20 | 61 | 74 | 3 | 86 | 91 | 92 | 93 | 18 |
| (Example 2) | | | | | | | | | | |
| OGA 3 L/ha | 0 | 0 | 0 | 0 | 0 | 55 | 71 | 71 | 59 | 4 |
| (Example 2) | | | | | | | | | | |
| OGA 2 L/ha | 15 | 10 | 9 | 5 | 0 | 20 | 15 | 9 | 0 | 0 |
| (Example 1) | | | | | | | | | | |
| MOSPILAN 20 | 0 | 0 | 0 | 26 | 2 | 0 | 44 | 51 | 68 | 21 |
| EC (active | | | | | | | | | | |
| substance: | | | | | | | | | | |
| Acetamiprid, | | | | | | | | | | |
| manufacturer: | | | | | | | | | | |
| Sumi Agro) | | | | | | | | | | |
| | Effectiveness (%) of the preparations in pepper crops | | | | | | | | | |
| CONTROL | — | — | — | — | — | — | — | — | — | — |
| OGA 2 L/ha | 15 | 0 | 46 | 24 | 27 | 15 | 3 | 38 | 6 | 0 |
| (Example 2) | | | | | | | | | | |
| OGA 3 L/ha | 22 | 0 | 100 | 18 | 86 | 30 | 58 | 59 | 23 | 19 |
| (Example 2) | | | | | | | | | | |
| OGA 2 L/ha | 20 | 0 | 0 | 18 | 14 | 0 | 0 | 20 | 15 | 16 |
| (Example 1) | | | | | | | | | | |
| MOSPILAN 20 | 0 | 0 | 0 | 11 | 70 | 0 | 64 | 83 | 70 | 56 |
| EC (active | | | | | | | | | | |
| substance: | | | | | | | | | | |
| Acetamiprid, | | | | | | | | | | |
| manufacturer: | | | | | | | | | | |
| Sumi Agro) | | | | | | | | | | |

### Conclusions:

OGA effectively controls spider mites and thrips in vegetable crops, the action of OGA is both direct on the pest and indirect. A repeat application of OGA increases or maintains high effectiveness of pest control. The effectiveness of OGA in controlling spider mites and thrips is comparable to that of chemical plant protection products.

Experiments were conducted to check the effectiveness of OGA in controlling aphids in vegetable cultivation and apple horticulture.

The effectiveness of aphid control in cabbage crops after the first application (T1+3, T1+7) of the OGA preparation ranged from 66% for the dose of 2 L/ha to 72% for the dose of 3 L/ha. After repeat application 2, the effectiveness of the OGA preparation increased and was more than 80% for the dose of 2 L/ha within 7 days after application 2, and 14 and 21 days after application 2 the effectiveness increased to almost 100%. The effectiveness of the OGA preparation in controlling aphids on cabbage was comparable to that of a synthetic plant protection product, Karate Zeon. The results are presented in Table 12.

TABLE 12
Effectiveness in controlling aphids
(Brevicoryne brassicae) in cabbage crops

| | Aphid control effectiveness (%) | | | | |
|---|---|---|---|---|---|
| COMBINATION | T1 + 3 | T1 + 7 | T2 + 7 | T2 + 14 | T2 + 21 |
| CONTROL | — | — | — | — | — |
| OGA 2 L/ha | 66 | 67 | 84 | 95 | 97 |
| (Example 2) | | | | | |
| OGA 3 L/ha | 72 | 72 | 79 | 89 | 92 |
| (Example 2) | | | | | |
| KARATE ZEON | 80 | 79 | 87 | 91 | 97 |
| 0.05% | | | | | |
| (active substance: | | | | | |
| lambda-cyhalothrin, | | | | | |
| manufacturer: | | | | | |
| Syngenta) | | | | | |

The application of OGA at the dose of 3 L/ha reduced the population of aphids feeding on carrot roots, which led directly to the reduction in the number of plants damaged by this pest. Tested parameter values in combination with OGA 3 L/ha were lower than in combination with the application of the Proteus 110 the chemical product. The results are presented in Table 13.

TABLE 13
Effect of OGA on carrot root aphids - (Pemphigus phenax)..

| | Number of plants | % of infested plants |
|---|---|---|
| | damaged by wooly | after treatments in |
| COMBINATION | aphid/3 m² | a 3 m² area |
| CONTROL | 53.7 | 28.4 |
| OGA 2 L/ha (Example 2) | 40.2 | 26.2 |
| OGA 3 L/ha (Example 2) | 35.5 | 20.9 |
| Proteus 110, (active | 46.2 | 26.3 |
| substance: thiacloprid, | | |
| deltamethrin, | | |
| manufacturer: Bayer) | | |

An experiment was conducted to determine the effect of the OGA preparation on the apple aphid in the apple horticulture. The effectiveness of the OGA preparation applied foliarly in 2 L/ha and 3 L/ha doses in controlling aphids was 60-67% 7 days after treatment (T1+7). The results are presented in Table 14.

TABLE 14
Effectiveness of OGA in controlling apple
aphids (Aphis pomi) in the apple tree.

| | Average number of aphids per | | | Control | |
|---|---|---|---|---|---|
| | 1 shoot | | | effectiveness (%) | |
| COMBINATION | PRE-T | T1 + 2 | T1 + 7 | T1 + 2 | T1 + 7 |
| CONTROL | 212.6 | 221.9 | 191.3 | — | — |
| OGA 2 L/ha | 173.2 | 137.5 | 71.6 | 26 | 60 |
| (Example 3) | | | | | |
| OGA 3 L/ha | 185.1 | 144.2 | 49.8 | 23 | 67 |
| (Example 3) | | | | | |
| Mospilan 20 SP | 256.1 | 34.3 | 5.3 | 84 | 98 |
| (active substance: | | | | | |
| Acetamiprid, | | | | | |
| manufacturer: Sumi | | | | | |
| Agro) | | | | | |

The effectiveness of OGA according to the present invention as a pest control contact preparation was tested in a Petri dish experiment. Aphids were selected for the test. In the experiment, pepper leaves were taken from plants naturally inhabited by aphids. The leaves with the aphids were placed on dishes with moist tissue paper. Two applications of the tested preparations were made. Observations of the number of viable aphids were carried out 3 and 7 days after the first and second treatment (Table 15). Observations carried out 7 days after application 1 showed the effectiveness of 87% for the dose of 2 L/ha and 92% for the dose of 3 L/ha. After application 2, the effectiveness increased to 100%. The effectiveness of the OGA preparation according to the present invention was higher than that of OGA 2 L/ha (Example 1) and comparable to that of the chemical product.

TABLE 15
Effectiveness of the OGA contact activity in controlling
green peach aphid (Myzus Persicae) on pepper leaves

| | 3 days | 7 days | 3 days | 7 days |
|---|---|---|---|---|
| | after | after | after | after |
| | treatment | treatment | treatment | treatment |
| | 1 | 1 | 2 | 2 |
| COMBINATION | Control effectiveness (%) | | | |
| CONTROL | — | — | — | — |
| OGA 2 L/ha (Example 2) | 65 | 87 | 92 | 100 |
| OGA 3 L/ha (Example 2) | 94 | 94 | 96 | 100 |
| OGA 2 L/ha (Example 1) | 30 | 25 | 31 | 15 |
| MOSPILAN 20 EC | 95 | 94 | 100 | 100 |
| (active substance: | | | | |
| Acetamiprid, | | | | |
| manufacturer: Sumi | | | | |
| Agro) | | | | |

### Conclusions:

In cabbage crops and in the Petri dish test on pepper, the effectiveness of OGA was more than 80% and comparable to the that of a synthetic plant protection product, such as Karate Zeon and Mospilan 20EC. In carrot crops, the protective effect of the OGA preparation is comparable to that of the commercial chemical product Proteus 110.

OGA has a contact effect on pests (direct action), after contacting OGA, the pest dies (Table 15). OGA effectively controls aphids in field vegetable crops.

Further experiments to determine the possibility of using OGA to reduce the population of pests feeding on leaves and roots were conducted on cabbage and carrots.

OGA was applied foliarly in 2 L/ha and 3 L/ha doses and the effect of the OGA preparation on vegetable whitefly and diamondback moth larvae feeding on cabbage leaves was studied. In the case of whitefly larvae (Table 16), the effectiveness of the OGA preparation was 44% and was observed 14 days after treatment 2, while the effectiveness of OGA in controlling diamondback moth caterpillars (Table 17) occurred also 14 days after treatment 2. For the lower dose of the OGA preparation the effectiveness was 59%, while it was 65% for the higher dose. The observed effectiveness in controlling whitefly larvae was higher for the OGA preparation (T2+14) than for the Benevia reference preparation (Table 17).

TABLE 16
Effectiveness in controlling vegetable whitefly
(Aleyrodes proletella) larvae in white cabbage crops.

| | CONTROL EFFICACY (%) | | | |
|---|---|---|---|---|
| COMBINATION | T + 3 | T + 7 | T2 + 7 | T2 + 14 |
| CONTROL | — | — | — | — |
| OGA 2 L/ha (Example 3) | 0 | 0.7 | 12.4 | 44.0 |
| OGA 3 L/ha (Example 3) | 0 | 0 | 0 | 10.0 |
| OGA 2 L/ha (Example 1) | 0 | 0 | 0 | 0 |
| Benevia 100 OD (active | 70.2 | 56.7 | 46.4 | 0 |
| substance: cyantraniliprole, | | | | |
| manufacturer FMC | | | | |

TABLE 17
Effectiveness in controlling diamondback moth (Plutella
xylostella) caterpillars in white cabbage crops.

| | CONTROL EFFICACY (%) | | | |
|---|---|---|---|---|
| COMBINATION | T + 3 | T + 7 | T2 + 7 | T2 + 14 |
| CONTROL | — | — | — | — |
| OGA 2 L/ha (Example 2) | 7.0 | 5.2 | 0.0 | 59.0 |
| OGA 3 L/ha (Example 2) | 0.0 | 0.0 | 0.0 | 65.3 |
| OGA 2 L/ha (Example 1) | 0 | 0 | 0 | 0 |
| Benevia 100 OD (active | 97.7 | 92.4 | 79.2 | 93.2 |
| substance: cyantraniliprole, | | | | |
| manufacturer FMC | | | | |

The impact of the foliar application on the carrot roots infested by carrot fly larvae was assessed. The OGA formulations were tested in two doses of 2 and 3 L/ha, Proteus 110 was used as the chemical product (Table 18). The number of carrot roots damaged by the carrot fly was the lowest in combination with OGA at the dose of 2 L/ha. The effectiveness level of the OGA (2 L/ha) activity was similar to that of Proteus 110.

TABLE 18
Effectiveness of the preparations in controlling
the carrot fly (Chamaepsila rosae).

| | Average number of plants | |
|---|---|---|
| | damaged by larval | Effectiveness |
| COMBINATION | feeding/100 roots | (%) |
| CONTROL | 16.2 | — |
| OGA 2 L/ha (Example 3) | 8.5 | 48 |
| OGA 3 L/ha (Example 3) | 12.2 | 25 |
| OGA 2 L/ha (Example 1) | 13.0 | 20 |
| Proteus 110, (active | 7.2 | 55 |
| substance: thiacloprid, | | |
| deltamethrin, | | |
| manufacturer: Bayer) | | |

### Conclusions:

OGA controls larvae feeding on plant leaves and roots by stimulating plant defensive processes. OGA protects carrot roots from the root-eating larvae at the level of chemical plant protection products.

### Summary of the Experiments

The obtained results from the experiments on economically important plant crops showed that in reducing fungal diseases, the OGA preparation effectively reduces powdery mildew in vegetable crops, fungal diseases in wheat crops and fungi of the genus Alternaria. In reducing powdery mildew, the OGA preparation is more effective in sheltered crops than in field crops. In sheltered crops, the effectiveness of the OGA preparation is about 90%, and in field crops it is about 80%. In field crops, the effectiveness of OGA is higher in reducing powdery mildew than downy mildew. The OGA preparation applied prophylactically in winter wheat crops reduces the infestation of leaves and stalks by fungal diseases at 40-60%. High effectiveness in controlling fungal diseases in plant crops, in particular at the occurring of infestation, indicates the possibility of using the OGA preparation in plant protection programmes, to complement protective measures aimed at reducing the use of synthetic plant protection products. In addition, an increase in the effectiveness of the preparation after its re-application observed in the experiments also indicates indirect activity of the OGA preparation on the plant. The OGA preparation stimulates plant defence mechanisms which are triggered during the pathogen attack.

The effectiveness of the OGA preparation in controlling spider mites, thrips and aphids is high and oscillates at the level from 60 to 90%, the second repeat treatment with the preparation according to the present invention increases the effectiveness of the pest control by a dozen or so percent. The OGA preparation exhibits higher effectiveness in reducing thrips nymphs as compared to adult individuals, particularly after a repeat treatment. In the Petri dish test and field cabbage crops, the effectiveness in controlling aphids was over 90% and was comparable to that of the currently used chemical products. The effectiveness of OGA in reducing larval feeding is from 45 to 65%.

In controlling pests with a piercing-sucking apparatus, i.e. spider mites, thrips and aphids, the OGA preparation exhibits mainly contact (direct) activity on pests, in the case of whitefly larvae, diamondback moth caterpillars and carrot fly larvae that feed on leaves or roots, the OGA preparation acts indirectly on the pest by acting on the plant. The observed effectiveness of OGA on larvae and caterpillars, occurring after some time and after repeat application, allows to believe that the OGA preparation stimulates plant resistance. The OGA preparation activates plant processes and metabolic transformations which are responsible for the plant defence mechanism. An example of such a process is the production of secondary metabolites such as terpenoids which have repellent properties. The accumulation of repellent compounds in the tissues of the plant makes it less “tasty” for leaf-eating larvae.

### Example 8

### Evaluation of the OGA Preparation Effect on the Stimulation of the Plant Defence Response

A gene expression analysis by the RNA-seq method allows to identify genes that are expressed in a given phase of plant growth in a particular tissue. A bioinformatic analysis allows to identify genes whose expression is statistically significantly different in the treated object as compared to the control object. A gene expression analysis was performed in tomato leaves after applying the OGA preparation in the 1 L/ha dose (Example 2). The experiment was conducted under controlled conditions in a phytotron chamber. The first treatment was performed in phase 4 of the leaves and then 3 treatments were performed every 4 days. Then, 48 h after the last treatment, the leaves were collected from the control combination, where the plants were treated with distilled water, and from the combination with OGA, immediately after the harvest, the leaves were frozen at −80° C. until mRNA isolation. The isolated mRNA from the tomato leaves was subjected to sequencing by the NGS (next-generation sequencing) method. mRNA sequencing showed activation of the genes encoding compounds, i.e. proteins, enzymes and phytohormones involved in the plant defensive response. As a result of the bioinformatic analysis, 1760 genes that were statistically significantly increased under the influence of the OGA preparation compared to the control object and 1329 genes with a significantly reduced expression were identified. Table 19 presents a fragment of the bioinformatic analysis results for the genes with a statistically significant difference between control (expression value is 1) and the OGA preparation (Example 2), where the fold change of the gene expression was at least 3.

TABLE 19
Results of the gene expression analysis

| | Value of the gene | | |
|---|---|---|---|
| | expression change (the | | |
| | number of times the | | |
| Gene No. | expression has increased) | Gene Name | Name of the gene encoding protein |
| Solyc04g079450.3 | 7.27 | SPZ3_ARATH | Serpin-Z3 |
| Solyc02g085910.3 | 7.14 | LBD41_ARATH | LOB domain-containing protein 41 |
| Solyc11g042560.1 | 5.42 | ERF22_ARATH | Ethylene-responsive transcription |
| | | | factor ERF022 |
| Solyc03g093130.3 | 4.93 | XTH25_ARATH | Probable xyloglucan |
| | | | endotransglucosylase/hydrolase |
| | | | protein 25 |
| Solyc02g090770.1 | 4.47 | EF112_ARATH | Ethylene-responsive transcription |
| | | | factor ERF112 |
| Solyc02g064830.3 | 4.32 | GH33_ARATH | Indole-3-acetic acid-amido |
| | | | synthetase GH3.3 |
| Solyc11g018775.1 | 4.32 | PERX_NICSY | Lignin-forming anionic peroxidase |
| Solyc01g090300.3 | 4.26 | ERF99_ARATH | Ethylene-responsive transcription |
| | | | factor 13 |
| Solyc10g050970.1 | 4.25 | EF109_ARATH | Ethylene-responsive transcription |
| | | | factor ERF109 |
| Solyc03g093120.3 | 4.12 | XTH25_ARATH | Probable xyloglucan |
| | | | endotransglucosylase/hydrolase |
| | | | protein 25 |
| Solyc08g036640.3 | 4.11 | TIF5A_ARATH | Protein TIFY 5A |
| | | | {ECO: 0000303\|PubMed: 17499004} |
| Solyc08g007830.1 | 4.1 | DRE1F_ARATH | Dehydration-responsive element- |
| | | | binding protein 1F |
| Solyc01g098690.2 | 3.96 | RLP50_ARATH | Receptor-like protein 50 |
| | | | {ECO: 0000303\|PubMed: 18434605} |
| Solyc10g050960.2 | 3.87 | CRF4_ARATH | Ethylene-responsive transcription |
| | | | factor CRF4 |
| Solyc02g076640.1 | 3.87 | DOGL4_ARATH | Protein DOG1-like 4 |
| | | | {ECO: 0000303\|PubMed: 17065317} |
| Solyc03g093080.3 | 3.87 | XTH22_ARATH | Xyloglucan |
| | | | endotransglucosylase/hydrolase |
| | | | protein 22 |
| Solyc12g009015.1 | 3.86 | ENV_SIVMK | Envelope glycoprotein gp160 |
| Solyc06g054630.2 | 3.84 | ERF17_ARATH | Ethylene-responsive transcription |
| | | | factor ERF017 |
| Solyc08g080290.3 | 3.84 | ERF26_ARATH | Ethylene-responsive transcription |
| | | | factor ERF026 |
| Solyc01g005160.3 | 3.84 | PUB21_ARATH | U-box domain-containing protein |
| | | | 21 |
| Solyc03g111300.1 | 3.82 | C94B3_ARATH | Cytochrome P450 94B3 |
| | | | {ECO: 0000305} |
| Solyc03g097050.3 | 3.79 | CSLD2_ARATH | Cellulose synthase-like protein D2 |
| Solyc08g074705.1 | 3.79 | DAAF1_DROPS | Dynein assembly factor 1, |
| | | | axonemal homolog |
| Solyc08g068640.3 | 3.76 | SDC1_ORYSJ | Serine decarboxylase 1 |
| Solyc01g087590.3 | 3.76 | CAC1F_HUMAN | Voltage-dependent L-type calcium |
| | | | channel subunit alpha-1F |
| | | | {ECO: 0000305} |
| Solyc02g063540.2 | 3.74 | 1A13_SOLLC | 1-aminocyclopropane-1-carboxylate |
| | | | synthase 3 |
| Solyc05g055080.1 | 3.73 | RS14_BUCAK | 30S ribosomal protein S14 |
| | | | {ECO: 0000255\|HAMAP- |
| | | | Rule: MF_00537} |
| Solyc02g080850.1 | 3.73 | FB333_ARATH | Probable F-box protein At4g22030 |
| Solyc01g079200.3 | 3.72 | G2OX6_ARATH | Gibberellin 2-beta-dioxygenase 6 |
| Solyc07g054980.2 | 3.68 | MYB15_ARATH | Transcription factor MYB15 |
| | | | {ECO: 0000305} |
| Solyc03g083680.1 | 3.66 | PLSY1_THEMA | Glycerol-3-phosphate |
| | | | acyltransferase 1 |
| | | | {ECO: 0000255\|HAMAP- |
| | | | Rule: MF_01043} |
| Solyc01g109920.2 | 3.65 | ECP40_DAUCA | Embryogenic cell protein 40 |
| Solyc05g054250.1 | 3.65 | HIP33_ARATH | Heavy metal-associated |
| | | | isoprenylated plant protein 33 |
| | | | {ECO: 0000303\|PubMed: 21072340, |
| | | | ECO: 0000303\|PubMed: 23368984} |
| Solyc03g121340.1 | 3.64 | NIH_ARATH | DExH-box ATP-dependent RNA |
| | | | helicase DExH2 {ECO: 0000305} |
| Solyc10g008080.2 | 3.59 | ATL40_ARATH | RING-H2 finger protein ATL40 |
| Solyc03g111750.2 | 3.58 | ATG16_ARATH | Autophagy-related protein 16 |
| | | | {ECO: 0000305} |
| Solyc08g036620.3 | 3.55 | TIF5A_ARATH | Protein TIFY 5A |
| | | | {ECO: 0000303\|PubMed: 17499004} |
| Solyc06g076060.1 | 3.53 | SMR3_ARATH | Cyclin-dependent protein kinase |
| | | | inhibitor SMR3 |
| | | | {ECO: 0000303\|PubMed: 24399300, |
| | | | ECO: 0000303\|PubMed: 26546445} |
| Solyc06g075690.3 | 3.51 | UFC_ARATH | Protein UPSTREAM OF FLC |
| Solyc06g062920.3 | 3.5 | Y1725_ARATH | Putative receptor-like protein kinase |
| | | | At1g72540 |
| Solyc12g005450.1 | 3.44 | Y1332_ARATH | Probable receptor-like protein |
| | | | kinase At1g33260 {ECO: 0000305} |
| Solyc02g094000.1 | 3.44 | CML19_ORYSJ | Putative calcium-binding protein |
| | | | CML19 |
| Solyc02g064980.1 | 3.43 | M3K20_ARATH | Mitogen-activated protein kinase |
| | | | kinase kinase 20 |
| | | | {ECO: 0000303\|PubMed: 21969089} |
| Solyc11g068390.1 | 3.43 | BIC1_ARATH | Protein BIC1 |
| | | | {ECO: 0000303\|PubMed: 27846570} |
| Solyc04g076730.1 | 3.42 | RAD18_SCHPO | Postreplication repair E3 ubiquitin- |
| | | | protein ligase rad18 |
| Solyc08g008280.3 | 3.41 | WRK53_ARATH | Probable WRKY transcription |
| | | | factor 53 |
| | | | {ECO: 0000303\|PubMed: 17369373} |
| Solyc08g036660.3 | 3.38 | TIF5A_ARATH | Protein TIFY 5A |
| | | | {ECO: 0000303\|PubMed: 17499004} |
| Solyc10g085700.1 | 3.34 | MAIL1_ARATH | Protein MAIN-LIKE 1 |
| | | | {ECO: 0000303\|PubMed: 24635680} |
| Solyc02g062550.3 | 3.33 | HSR4_ARATH | Protein HYPER-SENSITIVITY- |
| | | | RELATED 4 |
| | | | {ECO: 0000303\|PubMed: 15181213} |
| Solyc09g082870.2 | 3.31 | ACA13_ARATH | Putative calcium-transporting |
| | | | ATPase 13, plasma membrane-type |
| Solyc08g077610.2 | 3.29 | CLSY3_ARATH | SNF2 domain-containing protein |
| | | | CLASSY 3 |
| Solyc08g036625.1 | 3.27 | TIF5A_ARATH | Protein TIFY 5A |
| | | | {ECO: 0000303\|PubMed: 17499004} |
| Solyc01g079660.2 | 3.24 | A70_ARATH | Pathogen-associated molecular |
| | | | patterns-induced protein A70 |
| | | | {ECO: 0000303\|PubMed: 17215350} |
| Solyc08g066320.3 | 3.23 | SIK1_ORYSJ | LRR receptor-like serine/threonine- |
| | | | protein kinase SIK1 |
| | | | {ECO: 0000305} |
| Solyc06g035700.1 | 3.22 | ERF25_ARATH | Ethylene-responsive transcription |
| | | | factor ERF025 |
| Solyc09g011860.3 | 3.22 | OFT28_ARATH | O-fucosyltransferase 28 |
| | | | {ECO: 0000305} |
| Solyc08g068600.3 | 3.22 | SDC1_ORYSJ | Serine decarboxylase 1 |
| Solyc07g056000.3 | 3.21 | XTH16_ARATH | Probable xyloglucan |
| | | | endotransglucosylase/hydrolase |
| | | | protein 16 |
| Solyc10g011910.3 | 3.21 | WRK22_ARATH | WRKY transcription factor 22 |
| Solyc04g079360.1 | 3.2 | MYB77_ARATH | Transcription factor MYB77 |
| | | | (ECO: 0000303\|PubMed: 11597504} |
| Solyc06g074140.1 | 3.18 | PUB24_ARATH | E3 ubiquitin-protein ligase PUB24 |
| Solyc05g050210.3 | 3.18 | GDL61_ARATH | GDSL esterase/lipase At4g01130 |
| Solyc02g068680.1 | 3.18 | NRX1_ARATH | Probable nucleoredoxin 1 |
| Solyc03g119530.3 | 3.17 | LBD41_ARATH | LOB domain-containing protein 41 |
| Solyc10g080010.2 | 3.17 | MUC21_ARATH | Xylan glycosyltransferase MUCI21 |
| | | | {ECO: 0000305} |
| Solyc09g083360.3 | 3.16 | RS151_ARATH | 40S ribosomal protein S15-1 |
| Solyc01g073830.1 | 3.13 | PKL_ARATH | CHD3-type chromatin-remodeling |
| | | | factor PICKLE |
| Solyc01g065530.3 | 3.13 | COBL7_ARATH | COBRA-like protein 7 |
| Solyc04g074830.1 | 3.12 | CAP16_ARATH | Putative clathrin assembly protein |
| | | | At4g40080 |
| Solyc08g068680.3 | 3.12 | SDC1_ORYSJ | Serine decarboxylase 1 |
| Solyc07g051950.3 | 3.11 | SLAH1_ARATH | S-type anion channel SLAH1 |
| Solyc07g040710.3 | 3.1 | IQM5_ARATH | IQ domain-containing protein |
| | | | IQM5 {ECO: 0000305} |
| Solyc08g022240.1 | 3.1 | PLA15_ARATH | Phospholipase A1-Igammal, |
| | | | chloroplastic |
| Solyc03g007380.2 | 3.09 | WRK41_ARATH | Probable WRKY transcription |
| | | | factor 41 |
| Solyc11g071760.2 | 3.06 | CML37_ARATH | Calcium-binding protein CML37 |
| | | | {ECO: 0000303\|Ref. 5} |
| Solyc10g076240.2 | 3.06 | PER1_ARAHY | Cationic peroxidase 1 |
| Solyc03g116890.3 | 3.06 | WRK40_ARATH | Probable WRKY transcription |
| | | | factor 40 {ECO: 0000303\|Ref. 1} |
| Solyc03g005040.1 | 3.05 | CML31_ORYSJ | Probable calcium-binding protein |
| | | | CML31 |
| Solyc04g015360.3 | 3.04 | GATA8_ARATH | GATA transcription factor 8 |
| Solyc12g055710.1 | 3.04 | ATL60_ARATH | RING-H2 finger protein ATL60 |
| Solyc02g077060.2 | 3.04 | HR1_ARATH | RPW8-like protein 1 |
| | | | {ECO: 0000303\|PubMed: 11141561} |
| Solyc01g096720.3 | 3.01 | ZIFL1_ARATH | Protein ZINC INDUCED |
| | | | FACILITATOR-LIKE 1 |

### Conclusions:

The OGA preparation increased the expression of the genes involved in one of the main plant defensive responses within the systemic acquired resistance (SAR).

The SAR acquired resistance is induced by pathogens and natural substances mimicking the pathogen-plant interaction. The induction of SAR follows the signalling pathway of salicylic acid which is accompanied by the synthesis of signalling substances and pathogenesis-related proteins—PR (Pathogenesis-Related) plant proteins.

The expression of the genes encoding kinases from the MAPK (mitogen-activated protein kinase) family, which play a major role in the signal transmission within the SAR response, was increased in tomato leaves owing to the application of the OGA preparation. These kinases are activated in the cell when the cell, through receptors in the cell membrane, receives a signal that a pathogen attack has occurred, this signal being elicitors, i.e. fragments of the cell walls derived from the plant or pathogen, formed during the pathogen attack on the plant.

The OGA preparation according to the present invention, due to its unique structure, is able to mimic cell wall fragments and activates membrane receptors that activate the salicylic acid biosynthesis pathway, and indirectly the SAR immune response, using MAPK kinases. In addition to the genes encoding the kinases, the OGA preparation activated the genes directly involved in the synthesis of salicylic acid, i.e. the Dystrophin gene, UPF0182 protein Tery gene and WRKY DNA-binding transcription factor 70 gene. The OGA preparation increased the expression of Ethylene-responsive transcription factors, genes that are responsible for the transmission of signals in the cell under stress, namely the increased stress of diseases and pests. In addition to the above genes, the OGA preparation increased the expression of the genes responsible for the plant's response to injury: the lignin-forming anionic peroxidase gene and the Linoleate 13S-lipoxygenase 3-1 gene, increased the expression of the genes associated with the synthesis of proteins involved in the plant immune response, i.e. chloroplastic, Linoleate 13S-lipoxygenase 2-1, chloroplastic, 11-beta-hydroxysteroid dehydrogenase 1B, Respiratory burst oxidase homolog protein C, Probable 2-oxoglutarate-dependent dioxygenase JRG21, Probable aspartic proteinase GIP2, 11-beta-hydroxysteroid dehydrogenase A, Biotin synthase, Cationic peroxidase 1, including also genes encoding cell wall components. Cell wall sealing is one of the first processes in the plant response to a pathogen attack.

By increasing the expression of the genes related to plant immunity, OGA increases defensive capabilities of the plant by preparing it for stress, namely an attack of pathogens or pests. Additionally, the preparation has a contact effect on pests and exhibits effectiveness in reducing fungal diseases. OGA can be successfully used as a plant defence response stimulant and a natural agent reducing infestation by diseases and pests.

1. A method for obtaining a preparation based on oligogalacturonides (OGA) with a degree of polymerization DP from 2 to 10 by an enzymatic method, wherein water, a 40% tetrasodium ethylenediaminetetraacetate solution and sodium hydroxide are introduced into a reactor equipped with a stirrer and a heating system to obtain pH 12.0 of the reaction mixture, then fruit pectin is introduced into the reactor and the contents of the reactor are stirred until pectin has completely dissolved, then a 10% citric acid solution is added to obtain pH 5.0, then a 0.1% pectinolytic enzyme solution is introduced into the reactor, after which the contents of the reaction mixture is heated to reach the temperature of 80° C. which is maintained for another 30 minutes until the enzyme deactivates, then the reaction mixture is cooled to the temperature below 40° C. and a solution of potassium sorbate (1-5 kg of powder dissolved in a small amount of water) is added and stirred for another 30 minutes, the finished product in liquid form is poured into containers or spray-dried or lyophilized in order to obtain the product in solid form.

2. The method for obtaining the OGA preparation according to claim 1, characterized in that pectin is selected from citrus or apple pectin.

3. The method for obtaining the OGA preparation according to claim 1, characterized in that the pectinolytic enzyme is selected from the group comprising polygalacturonase or pectinase.

4. Use of the OGA preparation obtained according to claim 1 in agriculture or horticulture or vegetable cultivation, as a preparation having a plant biostimulating effect and/or a contact effect on pests.

5. The use according to claim 4 to stimulate plant defensive processes and enhance plant resistance to a pathogen and pest attack, especially in the cultivation of vegetables, horticultural plants and cereals.

6. The use according to claim 4 to reduce plant fungal diseases or to reduce infestation by pests.
